(19) United States
(12) Patent Application Publication
(10) Pub. No.: US 2001/0055569 A1
(43) Pub. Date: Dec. 27, 2001

(54) Nasal drug delivery composition

(75) Inventors: Stanley Stewart Davis, Nottingham (GB); Lisbeth Illum, Nottingham (GB)

Correspondence Address: AKIN, GUMP, STRAUSS, HAUER & FELD, L.L.P., ONE COMMERCE SQUARE, 2005 MARKET STREET, SUITE 2200, PHILADELPHIA, PA, 19103, US

(73) Assignee: West Pharmaceutical Services Drug Delivery & Clinical Research Centre, Ltd

(21) Appl. No.: 09/841,228

(22) Filed: Apr. 24, 2001

Related U.S. Application Data
(63) Continuation of application No. PCT/GB99/03489, filed on Oct. 21, 1999.

(30) Foreign Application Priority Data
Oct. 24, 1998 (GB) 9823246.5

(51) Int. Cl. A61K 9/00; A61K 35/78; A61K 31/192

(52) U.S. Cl. 424/43; 424/731; 514/568; 514/569

(57) ABSTRACT

The present invention provides a composition comprising an oil-in-water emulsion and a drug dissolved in the emulsion. The oil phase comprises a hydroxylated oil, particularly a hydroxylated vegetable oil. The preferred hydroxylated vegetable oil is castor oil.

### CROSS-REFERENCE TO RELATED APPLICATION

This application is a continuation of International Application No. PCT/GB99/03489, filed Oct. 21, 1999, the disclosure of which is incorporated herein by reference.

### BACKGROUND OF THE INVENTION

The present invention relates generally to a new composition for delivery of drugs to the nose for systemic absorption. More specifically, the present invention relates to an oil-in-water emulsion formulation for the delivery of poorly water soluble drugs, which need to be given in relatively high doses, to the nose for systemic absorption.

Examples of poorly water soluble drugs that are given in relatively high doses are analgesics, including non-steroidal anti-inflammatory drugs (NSAIDs), and anti-Parkinson drugs. By a relatively high dose of drug, we mean more than 1 mg of drug.

The nasal route of drug delivery affords rapid absorption of drugs into the blood circulation. In some cases the absorption of almost the whole dose can be achieved and the pharmacokinetics can be similar to intravenous administration. Such rapid and effective drug delivery can be useful in the treatment of crisis situations such as pain (to include breakthrough pain, headache), migraine, convulsions, impotence and nausea. Nasal formulations for the delivery of analgesic agents such as morphine, butorphanol, fentanyl, buprenorphine have been described. For a review, see Nasal Systemic Delivery, Eds. Chien et al. Dekker, New York, 1987.

The non-steroidal anti-inflammatory drugs (NSAIDs) such as the cyclooxygenase (COX) COX-1 and COX-2 inhibitors have an important role in pain management. Compounds include ibuprofen, flurbiprofen, diclofenac, indomethacin, piroxicam, ketoprofen, etodolac, diflusinal, meloxicam, aceclofenac, fenoprofen, naproxen, tiaprofenic acid and tolmetin. Such drugs are normally given by mouth for absorption from the gastrointestinal tract, but can also be given by other routes, which include injection.

Nasal delivery of poorly water soluble drugs that need to be given in a relatively high dose is often problematic. The maximum volume to be given in each nostril is 100 to 125 μl and with a low solubility of the drug, it is normally not possible to achieve a simple solution formulation. Moreover, the compounds can be irritant to mucosae.

It is known that solutions of non-steroidal anti-inflammatory drugs at relatively high concentrations can be prepared by the use of certain salt forms, e.g. K⁺, or by adjustment of pH. However, the osmolarity of such solutions can readily exceed isotonicity and, as a consequence, the solutions can be irritant.

WO-97/03659 describes the use of non-steroidal anti-inflammatory drugs (e.g. diclofenac or ibuprofen) for the treatment of nasal polyps, chronic rhinosinusitis or anosmia. There is no suggestion that the nasal route can be used for the systemic delivery of NSAIDs, nor is there a description of a two phase system, such as an emulsion, for this purpose.

EP-A-0524,587 describes the nasal administration of Ketorolac (U.S. Pat. No. 4,089,969) for analgesic and anti-inflammatory activity. Formulations based on the concepts of bioadhesion, e.g. using cellulose gums and block copolymers, as well as formulations containing enhancing agents are described. The use of an emulsion formulation was not described.

U.S. Pat. No. 5,707,644 describes the nasal delivery of NSAIDs and analgesics to the systemic circulation using small bioadhesive microspheres. There is no suggestion that a two phase liquid formulation, such as an emulsion, could be used.

Oil-in-water emulsion systems for the improved delivery of drugs via the nasal route have been described previously. Ko et al. (J. Microencaps. 15, 197, 1998) administered testosterone to rabbits. The drug was dissolved in soybean oil. Karali et al. (Pharm. Res. 9, 1024, 1992) used an oleic acid mono-olein emulsion to deliver a lipid soluble renin inhibitor. The emulsion was effective because it contained membrane modifying adjuvants. The use of a hydroxylated oil such as castor oil was not disclosed.

WO-93/12764 and GB-2,133,691 describe the potential use of emulsion systems for the nasal delivery of nicotine. The teaching of these two patents is to use systems of a defined viscosity and an oily emulsion is mentioned simply as a formulation option.

JP-4-173736 describes amphotericin containing emulsions and lyophilised counterparts based on soybean oil as the oily phase for use as nasal drops. It is well known that in such emulsions the drug is intercalated into the surface layer of the emulsion and is not dissolved in the oily phase (Davis et al. Ann N.Y. Acad. Sci. 507, 75, 1987)

JP-5-124965 describes the local treatment of nasal disorders using drugs dissolved in the oily phase of an emulsion. The oil phase was soybean oil and the drugs were steroid and steroid derivatives.

JP-7-258069 describes sustained release nasal drops containing vasoconstrictor and antihistamines in an oil-in-water emulsion for local effect.

Emulsion vehicles have also been used to improve the nasal delivery of polar drugs such as peptides and as vaccine adjuvants. In such formulations, the drug is not dissolved in the oil phase of the emulsion, but can be adsorbed to the surface of the emulsion droplets (WO-95/11700, U.S. Pat. No. 5,514,670, WO-93/05805, U.S. Pat. No. 5,716,637).

U.S. Pat. No. 5,179,079 describes the use of emulsions to disperse absorption promoting agents such as phospholipids.

In none of these prior art documents have oil-in-water emulsions based on hydroxylated oils, such as castor oil, been used for the nasal administration of drugs in order to provide for solubilisation of the therapeutic agent and reduced nasal irritation.

### BRIEF SUMMARY OF THE INVENTION

The present applicant has developed an oil-in-water formulation that can provide for the effective nasal delivery of drugs which are poorly soluble in water, such as analgesics, including NSAIDs, and drugs for the treatment of Parkinson's disease and impotence. The composition may also demonstrate a reduced nasal irritation.

According to the present invention, there is provided a pharmaceutical composition comprising (i) an oil-in-water emulsion and (ii) a drug other than a cannabinoid dissolved in the emulsion, wherein the oil phase comprises a hydroxylated oil, particularly a hydroxylated vegetable oil.

### DETAILED DESCRIPTION OF THE INVENTION

The composition of the invention can provide for the delivery of poorly water soluble drugs, which are given in a relatively high dose, to the nasal mucosa for subsequent delivery to the systemic circulation. By a poorly water soluble drug we mean a drug with a solubility in water less than 10 mg/ml at pH 7.4 at 25° C. By a relatively high dose of drug we mean more than 1 mg of drug.

The drug, which in a preferred embodiment is a poorly water soluble drug, is preferably largely contained within the oil phase of the oil-in-water emulsion. By “largely” we mean that more than one half, i.e. more than 50%, and preferably more than 75% of the available drug is dissolved in the oil phase on a weight basis.

The hydroxylated oil which is contained in the composition of the invention provides for solubilisation of the drug and can provide for effective solubilisation of poorly water soluble drugs so that a therapeutically relevant dose can be delivered via the nose. Moreover, the emulsion formulation can greatly reduce any irritation associated with the drug. Without wishing to be bound by any theory, it is believed that such reduction in irritation is due to the fact that the drug is dissolved largely in the oil phase, since it is the drug in the aqueous phase that can lead to irritation of the nasal mucosa.

By a hydroxylated oil we mean an oil that contains hydroxylated fatty acids. Preferred hydroxylated oils are hydroxylated vegetable oils, and a preferred hydroxylated vegetable oil for use in the present composition is castor oil.

Castor oil consists of the glycerides of ricinoleic acid which is a hydroxy fatty acid. By castor oil we include ricinus oil, oil of Palma Christie, tangantargon oil and Neoloid as described in the Merck Index 12th Edition p. 311. Castor oil is a fixed oil usually obtained by the cold pressing of the seeds of Ricinus Communis L., (Fam. Euphorbiaceae). The fatty acid composition is stated in the Merck Index to be 87% ricinoleic acid, 7% oleic acid, 3% linoleic acid, 2% palmitic acid, 1% stearic acid and dihydroxystearic acid in trace amounts.

We also include the oil from Ricinus Zanzibarinus in our definition of castor oil. This oil also has a high content of glycerides of ricinoleic acid (Evans, in Trease and Evans, Pharmacognosy, 13th Edition, Bailliere Tindall, London 1989, P. 333).

Conventional vegetable oils, such as soy bean oil, cotton seed oil and arachis oil, used for the preparation of pharmaceutical emulsions do not demonstrate such good drug solubility. The advantages of castor oil could be due to the fact that it contains hydroxylated fatty acids.

The oil phase in the emulsion can constitute from 1 to 50% v/v of the emulsion. A preferred concentration of oil in the emulsion is from 10 to 40% v/v and an especially preferred concentration is from 20 to 30% v/v.

A wide variety of drugs, other than cannabinoids, can be included in the composition of the invention. Suitable drugs not only include analgesic agents, such as NSAIDs, and drugs for the treatment of Parkinson's disease, but also drugs where rapid onset of action may be required, such as drugs for the treatment of nausea and vertigo, convulsions, panic attacks, cardiac problems, impotence, erectile dysfunction, migraine, sedation (particularly in children) and withdrawal symptoms. Suitable drugs may also include benzodiazapines, midazolam, diazepam and diamorphine.

Suitable non-steroidal anti-inflammatory drugs (NSAIDs) include the cyclooxygenase (COX) COX-1 and COX-2 inhibitors. Specific compounds which may be used in the compositions of the invention include ibuprofen, flurbiprofen, diclofenac, indomethacin, piroxicam, ketoprofen, etodolac, diflusinal, meloxicam, aceclofenac, fenoprofen, naproxen, tiaprofenic acid and tolmetin. Preferred compounds are ibuprofen and flurbiprofen.

The loading of the drug in the emulsion will be determined by the dose of the drug required for a therapeutic effect and the solubility of the drug in the hydroxylated oil. Doses of 10 mg to 100 mg could be administered. Some drugs may be oily in nature and thereby be miscible with the hydroxylated oil.

Typically, the drug is comprised in the emulsion at a concentration of from 0.1 to 20% w/v, preferably from 1 to 10% w/v, i.e. from 0.1 to 20, preferably from 1 to 10 g of drug in 100 ml of oil.

The nasal administration of analgesics using the compositions of the invention may provide for direct access to sites of action such as the cerebrospinal fluid and the nervous ganglia associated with conditions such as migraine. Consequently, the required dose of a NSAID administered nasally may be less than that required when given by the usual oral route.

In addition, the fact that some drugs will be mostly dissolved in the oil phase and, therefore, not in contact with water, may also help improve the stability of the drug in the formulation.

The emulsion compositions of the invention can be prepared using conventional methods such as by homogenisation of a mixture of the oil and drug with an aqueous phase, optionally together with a stabilizing agent. A microfluidizer or ultrasonic device can be used; the former is preferred for large scale production.

Where a stabilizer/emulsifier is used in the formation of the emulsion, it should be one which confers good stability to the emulsion and is pharmaceutically acceptable.

One suitable stabilizer is a block copolymer containing a polyoxyethylene block, i.e. a block made up of repeating ethylene oxide moieties. A suitable stabilizer of this type is Poloxamer, i.e. a polyoxyethylene-polyoxypropylene block copolymer, such as Poloxamer 188. See the Handbook of Pharmaceutical Excipients, p.352, 2nd Edn. Pharmaceutical Press, London, 1994, Eds, Wade and Weller.

A preferred stabilizer is a phospholipid emulsifier. Preferred phospholipids are the soy and egg lecithins, and egg lecithins, such as the material provided by Lipoid (Germany) known as Lipoid E80, which contains both phosphatidylcholine and phosphatidyl ethanoline, are particularly preferred. Other phospholipid materials could also be used including phospholipid-polyethylene glycol (PEG) conjugates (PEGylated phospholipids) that have been described for use in liposome systems, e.g. by Litzinger et al, Biochem Biophys Acta, 1190 (1994) 99-107.

The concentration of stabilizer/emulsifier can be from 0.1 to 10% w/v in the aqueous phase of the emulsion, i.e. from 0.1 to 10 g of stabilizer per 100 mls of the aqueous phase, but a preferred concentration is from 1 to 5% w/v.

The stability of the emulsion can be further enhanced by the addition of a pharmaceutically acceptable co-emulsifier. Suitable co-emulsifiers include the fatty acids and salts thereof and bile acid and salts thereof. Suitable fatty acids are those having greater than 8 carbon atoms in their structure with oleic acid being a preferred material. A preferred bile acid is deoxycholic acid. Suitable salts are the pharmaceutically acceptable salts such as the alkali metal, e.g. Na and K, salts. These co-emulsifiers can be added at a concentration of 1% w/v or less on the aqueous phase, i.e. 1 g or less of co-emulsifier per 100 mls of the aqueous phase of the emulsion. Bile salts and oleic acid are preferred co-emulsifiers.

The composition of the present invention may be adjusted, if necessary, to approximately the same osmotic pressure as that of the body fluids. This may be desirable where the composition is to be applied to delicate tissue membranes, such as those found in the nasal cavity. For example, compositions comprising NSAIDs can exceed isotonicity, becoming hypertonic. A composition which has been adjusted in this manner is said to be isotonic and will tend not to swell or contract the tissues with which it comes into contact and will result in minimal discomfort on application. The formation of isotonic solutions can be achieved by adding an ionic compound to the composition such as sodium chloride, or by adding glycerol.

It may also be appropriate to include buffering agents in the composition. For example, a buffer may be needed to maintain a pH that is compatible with nasal fluid, to ensure emulsion stability or to ensure that the drug does not partition from the emulsion oil phase into the aqueous phase.

It will be clear to the person skilled in the art that additional formulation components can be added to the emulsion. These could include agents that promote the transmucosal absorption of drugs such as surfactants, as well as thickening agents and gelling agents that will serve to retain the formulation in the nasal cavity for an extended period of time. Suitable thickening and gelling agents include cellulose polymers, particularly sodium carboxymethyl cellulose, alginates, gellans, pectins, acrylic polymers, agar-agar, gum tragacanth, gum xanthan, hydroxyethyl cellulose, chitosan, as well as block copolymers of the polyoxyethylene-polyoxypropylene class known as the poloxamers and poloxamines. Preservative agents such as methyl parabenzoates, benzylalcohol and chlorobutanol could also be added.

The emulsion can be administered to the nasal cavity using conventional nasal spray devices. These devices can be single dose or multiple dose systems. Such devices can be obtained from companies such as Pfeiffer and Valois.

The present invention is now illustrated but not limited with reference to the following examples.

### EXAMPLE 1

### Solubility of Flurbiprofen in Vegetable Oils

The solubility of flurbiprofen in different vegetable oils was measured by the addition of increasing quantities of the drug to an oil system and determination of the maximum amount that will dissolve by observation of the resultant solution and the onset of a cloudy nature or precipitation. The solubility of flurbiprofen (obtained from The Boots Co. Ltd.) at room temperature measured in this way was less than 50 mg/ml in soybean oil BP (obtained from Kahlshams, Sweden) and 150 mg/ml in castor oil BP (William Ransom, UK).

### EXAMPLE 2

### An Oil in Water Emulsion Containing 45 mg/ml Flurbiprofen and a 30% v/v Oil Phase was Prepared as Follows

Approximately 60 ml of castor oil BP (William Ransom, UK) was warmed to 30-40° C. and 11.25 g of flurbiprofen (Boots Co., UK) was then added and the mixture stirred to dissolve. The volume of the flurbiprofen solution was adjusted to 75 ml by adding further castor oil.

Phosphate buffered saline (PBS) solution (pH 7.4) was prepared by dissolving a PBS tablet (Sigma, UK) in 200 ml of water. 150 ml of this solution was warmed to 40° C. and 3.0 g of egg yolk lecithin (Lipoid E80, Leopold, Germany) was added and mixed to disperse. To the egg yolk phospholipid dispersion was added 4.2 g of glycerol (Boots Co. Ltd.) to maintain isotonicity. This mixture was then added to the flurbiprofen/castor oil solution and the two phases mixed using a Silverson L4R homogeniser, pulsed between speeds 5 and 10 for a period of 1 minute. This coarse emulsion was then passed three times through a Rannie Mini-Lab valve homogeniser at 10,000 psi to produce a milky off-white emulsion. The emulsion had a fine particle size (about 200 nm as measured using the method of photon correlation spectroscopy) and was stable on storage at room temperature. There was no evidence of separation of free oil nor drug crystals as viewed under a light microscope.

### EXAMPLE 3

### Irritation Test in Human

In order to evaluate the relative irritancy of a solution and emulsion formulation of flurbiprofen two different formulations were evaluated.

Solution formulation: Flurbiprofen as the potassium salt was dissolved in water at a concentration of 45 mg/ml and the resulting solution administered to the nose using a Pfeiffer multidose nasal device. A volume of 50 μl was administered into one nostril.

Emulsion formulation: An oil-in-water emulsion formulation as described in example 2 containing 45 mg/ml of flurbiprofen was prepared and filled into a Pfeiffer multidose nasal spray device. A dose of 50 μl was administered into one nostril.

One subject (female, age 50) tested each formulation on two separate occasions.

Irritancy was assessed using an analogue scale. The solution formulation based on the potassium salt of flurbiprofen was noted as being irritant at a value of 10 on the irritancy scale.

The emulsion formulation was less irritant, being assessed as 4 on the irritancy scale.

### EXAMPLE 4

### Solubility of Non-steroidal Drugs in Castor Oil and Soybean Oil

The solubility of additional NSAIDs in castor oil and soybean oil was measured at room temperature as in Example 1. Ibuprofen, indomethacin and naproxen (as obtained from Sigma Chemical Co.) were investigated. The results in Table 1 demonstrate the beneficial effect of a hydroxylated vegetable oil (namely castor oil) in improving drug solubility so that a nasal emulsion of low irritation can be formulated.

TABLE 1
The solubility of ibuprofen, indomethacin and naproxen in castor
oil (B.P) and soybean oil

| | Solubility in castor oil | Solubility in soybean oil |
|---|---|---|
| Drug | (mg/g) | (mg/g) |
| Ibuprofen | >100 | >30 < 40 |
| Indomethacin | >15 < 20 | <5 |
| Naproxen | >20 < 40 | <10 |
| Apomorphine | >30 < 50 | <10 |

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A pharmaceutical composition adapted for nasal administration comprising (i) an oil-in-water emulsion and (ii) a drug dissolved in the emulsion, wherein the oil phase comprises a hydroxylated oil and wherein the drug is for systemic delivery and is also an analgesic agent, a drug for the treatment of Parkinson's disease, a drug for the treatment of impotence or a non-steroidal anti-inflammatory drug (NSAID), other than a cannabinoid.

2. A composition adapted for nasal administration comprising (i) an oil-in-water emulsion and (ii) a drug dissolved in the emulsion, wherein the oil phase comprises a hydroxylated oil and wherein the drug is for systemic delivery and is also an analgesic agent, a drug for the treatment of Parkinson's disease, a drug for the treatment of impotence or a non-steroidal anti-inflammatory drug (NSAID), other than a cannabinoid, for use in medicine.

3. A composition according to claim 1, wherein the hydroxylated oil is a hydroxylated vegetable oil.

4. A composition according to claim 3, wherein the hydroxylated vegetable oil is castor oil.

5. A composition according to claim 1, wherein the NSAID is flurbiprofen.

6. A composition according to claim 1, wherein the NSAID is ibuprofen.

7. A composition according to claim 1, wherein the NSAID is a COX-1 or COX-2 inhibitor.

8. A composition according to claim 1, wherein more than 50% of the drug is dissolved in the oil phase on a weight basis.

9. A composition according to claim 8, wherein more than 75% of the drug is dissolved in the oil phase on a weight basis.

10. A method for the treatment of pain which comprises delivering an oil-in-water emulsion containing a systemically active drug other than a cannabinoid by the nasal route.

11. A method according to claim 1 0, wherein the drug is a NSAID.

12. The use of a composition according to claim 1, for the systemic delivery of a drug by the nasal route.

13. The use of a composition according to claim 1, in the manufacture of a medicament for nasal administration.

14. The use of a composition according to claim 1, in the manufacture of a medicament for systemic delivery of the drug by the nasal route.
